(19) United States
(12) Patent Application Publication
(10) Pub. No.: US 2025/0308712 A1
(43) Pub. Date: Oct. 2, 2025

(54) COMPLEX DISEASE MARKER DISCOVERY USING CUMULANTS AND ISING HAMILTONIANS

(71) Applicant: International Business Machines Corporation, Armonk, NY (US)

(72) Inventors: Aritra Bose, White Plains, NY (US); Aldo Guzman Saenz, White Plains, NY (US); Laxmi Parida, Mohegan Lake, NY (US); Daniel Enoch Platt, Putnam Valley, NY (US); Kahn Rhrissorrakrai, Middle Village, NY (US); Filippo Utro, Pleasantville, NY (US)

(21) Appl. No.: 18/621,167

(22) Filed: Mar. 29, 2024

(51) Int. Cl. G16H 70/60; G06N 5/04

(52) U.S. Cl. CPC G16H 70/60; G06N 5/04

(57) ABSTRACT

A method for capturing distinct higher order interactions of a dataset relevant to biological inferences includes deriving Hamiltonian parameters for the dataset, wherein the dataset includes data responsive to a phenotype of interest. The method further includes generating a partition function responsive to the Hamiltonian parameters; calculating cumulant moments of the partition function and deriving higher order cumulants using the Hamiltonian parameters, wherein the higher order cumulants are responsive to the phenotype of interest.

### BACKGROUND

The present invention generally relates to the discovery of complex disease markers, and more particularly, to the discovery of complex disease markers using Cumulants and Ising Hamiltonians.

It is known that genetic alterations can impact the response a patient has to a drug response therapy. Identifying which genetic alterations impact which drug response therapy remains a challenging task since only several types of genetic alterations have been identified and validated. As such, patient population risk stratification is a critical element for understanding the etiology and epidemiology of a disease. Often researchers either leverage prior biological knowledge to study suspected risk factors of a given disease to distinguish sub-populations, or researchers look for correlations of features to some target phenotypes in order to discover risk factors that may serve as disease markers. Yet, most diseases and syndromes represent a highly complex set of interactions between different biological entities, potentially operating across biological scales, from a single cell to an entire organ system.

### SUMMARY

A method for capturing distinct higher order interactions of a dataset relevant to biological inferences includes deriving Hamiltonian parameters for the dataset, wherein the dataset includes data responsive to a phenotype of interest and generating a partition function responsive to the Hamiltonian parameters. The method further includes calculating cumulant moments of the partition function and deriving higher order cumulants using the Hamiltonian parameters, wherein the higher order cumulants are responsive to the phenotype of interest.

Embodiments of the invention are also directed to computer-implemented methods and computer program products having substantially the same features and functionality as the computer system described above.

Additional technical features and benefits are realized through the techniques of the present invention. Embodiments considered a part of the claimed subject matter. For a better understanding, refer to the detailed description and to the drawings.

### BRIEF DESCRIPTION OF THE DRAWINGS

The specifics of the exclusive rights described herein are particularly pointed out and distinctly claimed in the claims at the conclusion of the specification. The foregoing and other features and advantages of the embodiments of the invention are apparent from the following detailed description taken in conjunction with the accompanying drawings in which:

FIG. 1 shows a flow diagram illustrating a general overall method for capturing distinct higher order interactions relevant to a biological inference, according to an embodiment.

FIG. 2 is a flow diagram illustrating a current method for processing a large dataset using cumulants.

FIG. 3A is a flow diagram illustrating a method for capturing distinct higher order interactions of a dataset relevant to a biological inference through the calculation of cumulants using the partition function of the Ising model, according to an embodiment.

FIG. 3B is a flow diagram illustrating a method for capturing distinct higher order interactions of a dataset relevant to a biological inference through the calculation of cumulants using the partition function of the Ising model, according to another embodiment.

FIG. 4 shows a block diagram of an example computer system for use in accordance with one or more embodiments of the invention.

FIG. 5 is an operational block diagram illustrating a method for capturing distinct higher order interactions of a dataset relevant to a biological inference through the calculation of cumulants using the partition function of the Ising model, according to an embodiment.

### DETAILED DESCRIPTION

In one embodiment of the invention, a method for capturing distinct higher order interactions of a dataset relevant to biological inferences includes deriving Hamiltonian parameters for the dataset, wherein the dataset includes data responsive to a phenotype of interest and wherein the Hamiltonian parameters include Hamiltonian parameter covariances. The method further includes generating a partition function responsive to the Hamiltonian parameters, calculating cumulant moments and complex roots of the partition function and deriving higher order cumulants using the Hamiltonian parameters, wherein the higher order cumulants are responsive to the complex roots and the phenotype of interest and include cumulant covariances. An embodiment of the method allows for biological insight to be derived from both the parameter values of the Hamiltonian parameters and from other specific cumulants. Additionally, this further allows for using linear regression from data to obtain the Hamiltonian parameters which include higher-order interactions relevant to biological inferences.

In some examples of the method, the phenotype of interest includes genetic alterations which impact drug response therapy. An embodiment of the method allows for gaining biological insight on specific phenotypes of interest using Hamiltonian parameters which include higher-order interactions that are relevant to biological inferences.

In further examples of the method, the Hamiltonian parameters are derived using a Machine Learning/Artificial Intelligence Regression model. An embodiment of the method allows using a Machine Learning/Artificial Intelligence Regression model to develop learnable parameters from the Hamiltonian parameters to identify cumulants.

In yet further examples of the method, the Hamiltonian parameters are derived by computing moments of the dataset and applying an optimization procedure to the moments. An embodiment of the method allows for the derivation of Hamiltonian parameters which include higher-order interactions that are relevant to biological inferences to be determined by using and optimizing the moments of a dataset.

In yet further examples of the method, the optimization procedure is Newton-Raphson method. An embodiment of the method allows the Newton-Raphson method to be used to optimize moments of a dataset which are used to determine Hamiltonian moments.

In yet further examples of the method, generating a partition function includes generating the partition function from the complex roots of the partition function, wherein the partition function is responsive to the Hamiltonian parameters. An embodiment of the method allows for the partition function to be represented in terms of its roots which further allows for cumulants which describe biological state variable interactions to be determined.

In yet further examples of the method, wherein calculating cumulant moments of the partition function includes calculating 3^{rd} order cumulants of the partition function, and wherein deriving the higher order cumulants includes deriving 4^{th} order cumulants and higher. An embodiment of the method allows for the 3^{rd} and higher order cumulants to be more easily derived, where the 3^{rd} order cumulant is equal to the central moment of the distribution. In yet further examples of the method, evaluating a statistical significance of the Hamiltonian parameters and cumulants, wherein the statistical significance is used to identify higher order interactions relevant to biological inferences, to limit combinations of the biological inferences of interest and to indicate when a result exceeds a predefined threshold parameter. An embodiment of the method allows for the statistical significance of the Hamiltonian parameters and cumulants to be used to identify higher order interactions relevant to biological inferences, to limit combinations of the biological inferences of interest and to indicate when a result exceeds a predefined threshold parameter.

In another aspect of the invention, a computing system including a processor configured to perform operations for capturing distinct higher order interactions of a dataset relevant to a biological inference where the operations include deriving Hamiltonian parameters for the dataset, wherein the dataset includes data responsive to a phenotype of interest and Hamiltonian parameter covariances, and generating a partition function responsive to the Hamiltonian parameters. The method further includes calculating cumulant moments and complex roots of the partition function and deriving higher order cumulants using the Hamiltonian parameters, wherein the higher order cumulants are responsive to the complex roots and the phenotype of interest and include cumulant covariances. An embodiment of the computer system is configured to perform operations that allow for biological insight to be derived from both the parameter values of the Hamiltonian parameters and from other specific cumulants. Additionally, this further allows for using linear regression from data to obtain the Hamiltonian parameters which include higher-order interactions relevant to biological inferences.

In some examples of the computing system, the phenotype of interest includes genetic alterations which impact drug response therapy. An embodiment of the computer system is configured to perform operations for gaining biological insight on specific phenotypes of interest using Hamiltonian parameters which include higher-order interactions that are relevant to biological inferences.

In further examples of the computing system, the Hamiltonian parameters are derived using a Machine Learning/Artificial Intelligence Regression model. An embodiment of the computer system is configured to use a Machine Learning/Artificial Intelligence Regression model to develop learnable parameters from the Hamiltonian parameters to identify cumulants.

In yet further examples of the computing system, the Hamiltonian parameters are derived by computing moments of the dataset and applying an optimization procedure to the moments. An embodiment of the computer system is configured to derive Hamiltonian parameters which include higher-order interactions that are relevant to biological inferences to be determined by using and optimizing the moments of a dataset.

In yet further examples of the computing system, the optimization procedure is Newton-Raphson method. An embodiment of the computer system is configured to use the Newton-Raphson method to optimize moments of a dataset which are used to determine Hamiltonian moments.

In yet further examples of the computing system, generating a partition function includes generating the partition function from the complex roots of the partition function, wherein the partition function is responsive to the Hamiltonian parameters. An embodiment of the computer system is configured to allow the partition function to be represented in terms of its roots which further allows for cumulants which describe biological state variable interactions to be determined.

In yet further examples of the computing system, calculating cumulant moments of the partition function includes calculating 3^{rd} order cumulants of the partition function and deriving the higher order cumulants includes deriving 4^{th} order cumulants and higher. An embodiment of the computer system is configured to allow for the 3^{rd} and higher order cumulants to be more easily derived, where the 3^{rd} order cumulant is equal to the central moment of the distribution.

In yet further examples of the computing system, evaluating a statistical significance includes evaluating a statistical significance of the Hamiltonian parameters and cumulants, wherein the statistical significance is used to identify higher order interactions relevant to biological inferences, to limit combinations of the biological inferences of interest and to indicate when a result exceeds a predefined threshold parameter. An embodiment of the computer system is configured for the statistical significance of the Hamiltonian parameters and cumulants to be used to identify higher order interactions relevant to biological inferences, to limit combinations of the biological inferences of interest and to indicate when a result exceeds a predefined threshold parameter.

Yet another aspect of the invention includes a computer program product having a computer readable storage medium. The computer readable storage medium stores program instructions which are executable by a processor to cause the processor to perform operations for capturing distinct higher order interactions of a dataset relevant to a biological inference. The operations include the steps of deriving Hamiltonian parameters for the dataset, wherein the dataset includes data responsive to a phenotype of interest and wherein the Hamiltonian parameters include Hamiltonian parameter covariances. The method further includes generating a partition function responsive to the Hamiltonian parameters, calculating cumulant moments and complex roots of the partition function and deriving higher order cumulants using the Hamiltonian parameters, wherein the higher order cumulants are responsive to the complex roots and the phenotype of interest and include cumulant covariances. An embodiment of the computer program product is configured to perform operations that allow for biological insight to be derived from both the parameter values of the Hamiltonian parameters and from other specific cumulants. Additionally, this further allows for using linear regression from data to obtain the Hamiltonian parameters which include higher-order interactions relevant to biological inferences.

In some examples of the computer program product, the phenotype of interest includes genetic alterations which impact drug response therapy. An embodiment of the computer program product is configured to perform operations for gaining biological insight on specific phenotypes of interest using Hamiltonian parameters which include higher-order interactions that are relevant to biological inferences.

In some examples of the computer program product, the Hamiltonian parameters are derived using a Machine Learning/Artificial Intelligence Regression model. An embodiment of the computer program product is configured to use a Machine Learning/Artificial Intelligence Regression model to develop learnable parameters from the Hamiltonian parameters to identify cumulants.

In some examples of the computer program product, the Hamiltonian parameters are derived by computing moments of the dataset and applying an optimization procedure to the moments. An embodiment of the computer program product is configured to derive Hamiltonian parameters which include higher-order interactions that are relevant to biological inferences to be determined by using and optimizing the moments of a dataset.

In some examples of the computer program product, the optimization procedure is Newton-Raphson method. An embodiment of the computer program product is configured to use the Newton-Raphson method to optimize moments of a dataset which are used to determine Hamiltonian moments.

In some examples of the computer program product, generating a partition function includes generating the partition function from the complex roots of the partition function, wherein the partition function is responsive to the Hamiltonian parameters. An embodiment of the computer system is configured to allow the partition function to be represented in terms of its roots which further allows for cumulants which describe biological state variable interactions to be determined.

In some examples of the computer program product, calculating cumulant moments of the partition function includes calculating 3^{rd} order cumulants of the partition function and deriving the higher order cumulants includes deriving 4^{th} order cumulants and higher. An embodiment of the computer system is configured to allow for the 3^{rd} and higher order cumulants to be more easily derived, where the 3^{rd} order cumulant is equal to the central moment of the distribution.

In yet another aspect of the invention, a method for capturing distinct higher order interactions of a dataset relevant to a biological inference includes obtaining the dataset, wherein the dataset is responsive to a phenotype of interest and deriving Hamiltonian parameters for the dataset using a Machine Learning/Artificial Intelligence Regression model and wherein the Hamiltonian parameters include Hamiltonian parameter covariances. The method further includes generating a partition function responsive to the Hamiltonian parameters, calculating cumulant moments and complex roots of the partition function and deriving higher order cumulants using the Hamiltonian parameters, wherein the higher order cumulants are responsive to the complex roots and the phenotype of interest and include cumulant covariances. An embodiment of the method allows for biological insight to be derived from both the parameter values of the Hamiltonian parameters and from other specific cumulants using a Machine Learning/Artificial Intelligence Regression model.

In yet another aspect of the invention, a method for capturing distinct higher order interactions of a dataset relevant to a biological inference includes obtaining the dataset, wherein the dataset is responsive to a phenotype of interest and deriving Hamiltonian parameters for the dataset by computing moments of the dataset and applying an optimization procedure to the moments, wherein the Hamiltonian parameters include Hamiltonian parameter covariances. The method further includes generating a partition function responsive to the Hamiltonian parameters, calculating cumulant moments and complex roots of the partition function and deriving higher order cumulants using the Hamiltonian parameters, wherein the higher order cumulants are responsive to the complex roots and the phenotype of interest and include cumulant covariances. An embodiment of the method allows for biological insight to be derived from both the parameter values of the Hamiltonian parameters and from other specific cumulants by computing moments of the dataset and applying an optimization procedure to the moments.

As discussed briefly above, it is known that genetic alterations can impact the response a patient has to a drug response therapy. Therefore, identifying which genetic alterations impact which drug response therapy remains a challenging task since only several types of genetic alterations have been identified and validated. This task is made even more challenging as the genetic alterations are often hidden in large complex sequencing datasets. As such, patient population risk stratification is a critical element for understanding the etiology and epidemiology of a disease. Moreover, population level analyses are often limited due to incomplete molecular data for accessing the nano-scale and micro-scale entities. More typically, population level analyses include general level patient information that is contained in the Electronic Health Records (EHRs) of patients that represent some phenotypic measure that is the result of the myriad number of interactions used to create the EHR. Unfortunately, however, while these complex higher order interactions between variables may be distinguished using cumulants, the cumulant computational cost grows combinatorially. Accordingly, different approaches are required to expand their usage beyond 50 features and a 5^{th} order, which yields 95,344,200 joint cumulants.

Also, as discussed briefly above, identifying which genetic alterations impact drug response therapy remains a challenging task due to the fact that genetic alterations are often hidden in large complex sequencing datasets. And while the datasets that include the complex higher order interactions may be distinguished using cumulants, the cumulant computational cost tend to become very large. Accordingly, in order for cumulants to be useful in distinguishing which genetic alterations affect drug response therapy, an approach which is able to handle situations having greater than 50 features and a 5^{th} order cumulant (which yields 95,344,200 joint cumulants) is preferred. As is known, in probability theory and statistics, the cumulants of a probability distribution are a set of quantities that provide an alternative to the moments of the distribution. Thus, any two probability distributions whose moments are identical will have identical cumulants. The first order cumulant (i.e., first cumulant) is equal to the mean of the distribution, the second order cumulant (i.e., second cumulant) is equal to the variance of the distribution and the third order cumulant (i.e., third cumulant) is equal to the central moment of the distribution.

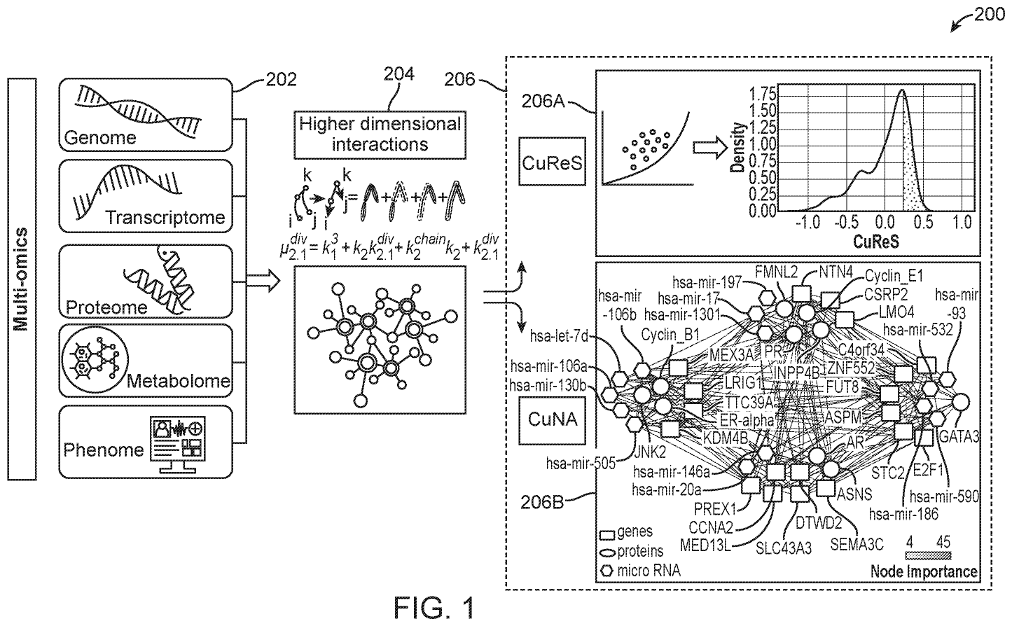

Referring to FIG. 1, a flow diagram illustrating an embodiment of a general overall method 200 for capturing distinct higher order interactions relevant to a biological inference is shown, and includes gathering a multi-nomic dataset 202 which may include data related to Genomes, Transcriptome, Proteome, Metabolome, Phenome, etc. The multi-nomic dataset 202 is then processed 204 to identify higher dimensional interactions (<fifth order cumulants) where the higher dimensional interactions may be further processed 206 using a Cumulant Based Network Analysis (CuNA) approach 206A or a Cumulant Based Drug Response (CuRes) Therapy approach 206B.

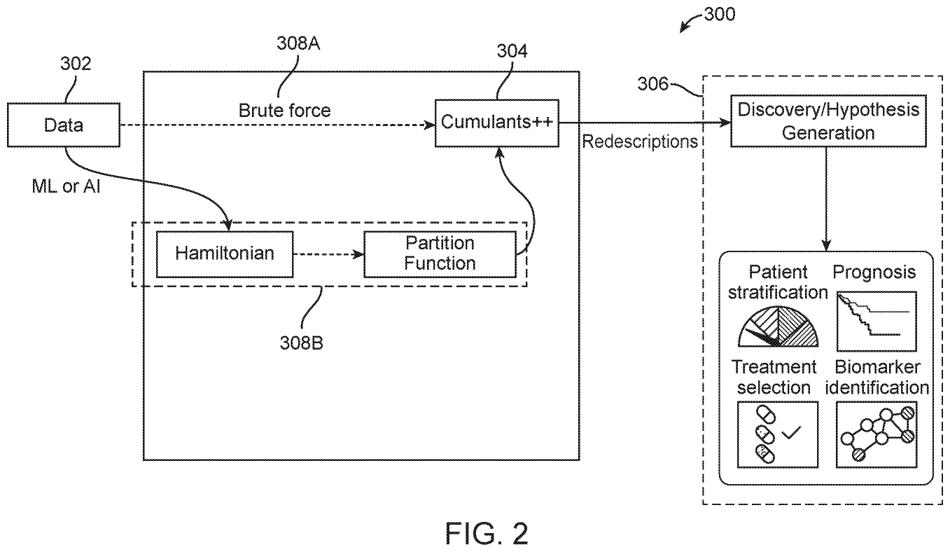

Referring to FIG. 2, a flow diagram illustrating a current method 300 for processing a large dataset 302 using cumulants 304 in order to determine a desired result 306, such as patient stratification, disease prognosis, treatment selection and biomarker identification, is shown. Currently, there are two ways to obtain the cumulants needed for the discovery of disease treatment/prognosis and/or the generation of a hypothesis regarding disease treatment/prognosis and they involve 1) a ‘brute force’ approach 308A, and 2) a Hamiltonian/Partition Function approach 308B. Unfortunately, both of these approaches are extremely labor intensive and result in a large cumulant computational cost. For example, regarding the ‘brute force’ approach 308A, as a 5^{th} order cumulant yields over 95 million joint cumulants, the complexity of computing 5^{th} order and higher cumulants becomes increasingly difficult. With regards to the Hamiltonian/Partition Function approach 308B, although determining the Hamiltonian parameters from the large dataset 302 is achievable, applying the Hamiltonian parameters to the partition function to determine the cumulants is a daunting task. Ultimately, no matter which of these two approaches is used, the task is extremely labor intensive and results in a large cumulant computational cost.

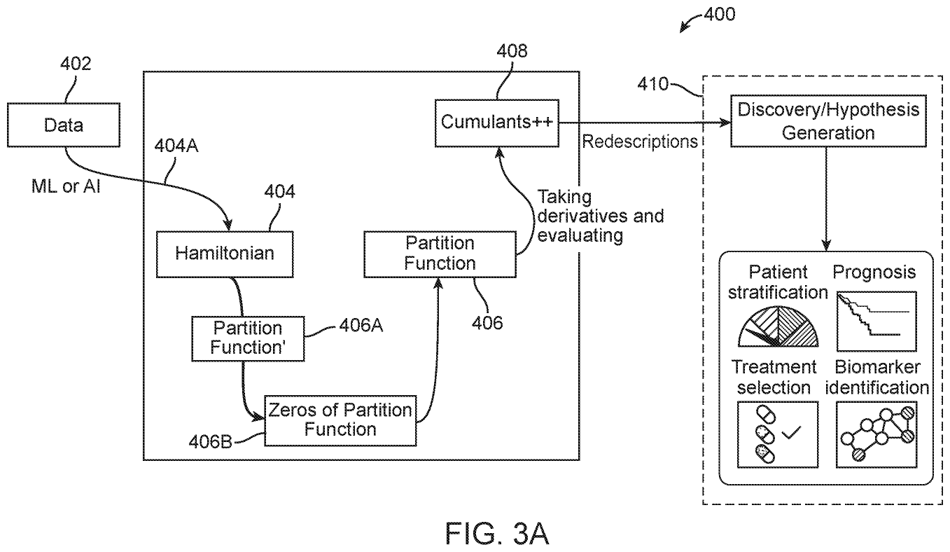

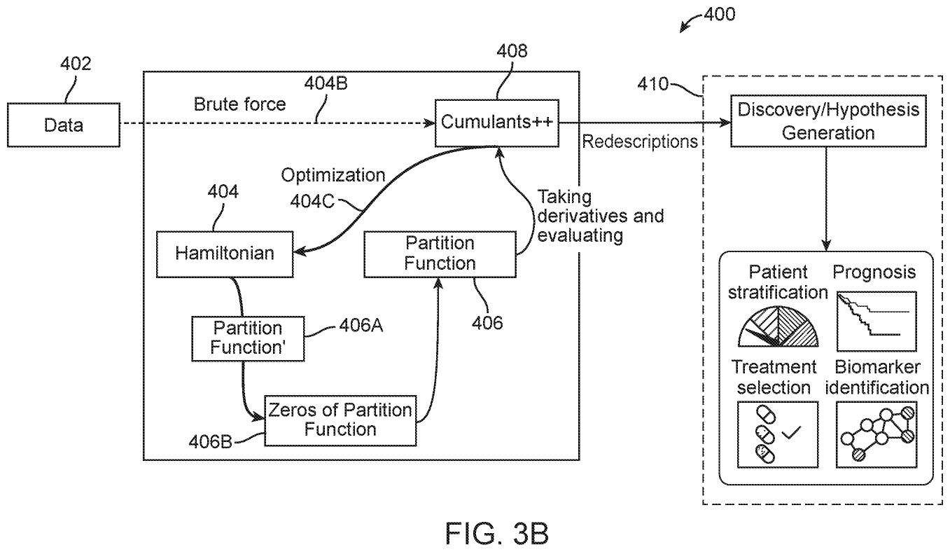

Referring to FIG. 3A, a flow diagram illustrating one embodiment of a method 400 for capturing distinct higher order interactions of a dataset relevant to a biological inference through the calculation of cumulants using the partition function of the Ising model is shown, where the partition function is a function of the Hamiltonian parameters. The Hamiltonian parameters embody a description of the interaction between biological state variables, wherein the derivatives of the log of the partition function yield the cumulants. The method 400 includes obtaining a dataset 402 representing the phenotype of interest, such as which genetic alterations impact drug response therapy. The Hamiltonian parameters 404 for the dataset 402 are then determined. This may be accomplished by using a Machine Language (ML)/Artificial Intelligence (AI)/Regression approach. The partition function 406 of the dataset 402 is determined by evaluating the partition function 406A of the Hamiltonian parameters 404 from its roots and then computing the zeros of the partition function (i.e., moments) 406B of the Hamiltonian parameters 404. The cumulants are then determined by taking the derivatives of the partition function 406, where the result is the combination of the Hamiltonian parameters and the cumulant 408. The Hamiltonian parameters and specific cumulants are then evaluated 410 to derive biological insight which can indicate the interacting features driving the phenotype of interest (e.g. therapeutic response, patient prognosis, disease risk, etc.).

Referring to FIG. 3B, a flow diagram illustrating another embodiment of a method 400 for capturing distinct higher order interactions of a dataset relevant to a biological inference through the calculation of cumulants using the partition function of the Ising model is shown, where the partition function is a function of the Hamiltonian parameters. The method 400 includes obtaining a dataset 402 representing the phenotype of interest, such as which genetic alterations impact drug response therapy. The Hamiltonian parameters 404 for the dataset 402 are then determined. This may be accomplished using an approach that includes a brute-force moments (i.e., third cumulant) computation with an optimization of the results of the brute-force moments computation using Newton's method (i.e., Newton-Raphson method). The partition function 406 of the dataset 402 is determined by evaluating the partition function of the Hamiltonian parameters 406A from its roots and then computing the zeros of the partition function (i.e., moments) 406B of the Hamiltonian parameters 404. The higher order cumulants (i.e., 4^{th} order and higher) are then determined by taking the derivatives of the partition function 406, where the result is the combination of the Hamiltonian parameters and the cumulant 408. The Hamiltonian parameters and specific cumulants are evaluated 410 to derive biological insight which can indicate the interacting features driving the phenotype of interest (e.g. therapeutic response, patient prognosis, disease risk, etc.).

The method of the invention provides a means for capturing distinct higher order interactions through the calculation of cumulants from Ising Hamiltonian parameters where “spins” define the biological system state. To achieve this-a number of other problems of possible general utility were solved, including defining directional partition functions taking scalar parameters, computing roots for this directional partition function, extracting directional cumulants from the directional partition function and constructing joint cumulants from linear combinations of directional cumulants.

It should be appreciated that the embodiments of the invention provide several distinct points of novelty, including a) the ability to learn parameters of the generating function (Hamiltonian) by using: Optimization procedure such as Newton-Raphson to compute Hamiltonian parameters from low order cumulants, b) biological insight is derived from both the parameter values of the Hamiltonian and the specific cumulants, c) obtaining Hamiltonians including higher-order interactions relevant to biological inferences using linear regression from data, d) extracting joint cumulants from directional derivative derived scalar cumulants and e) using directional partition function roots to extract directional scalar cumulants.

Various aspects of the present disclosure are described by narrative text, flowcharts, block diagrams of computer systems, and/or block diagrams of the machine logic included in computer program product (CPP) embodiments. With respect to any flowcharts, depending upon the technology involved, the operations can be performed in a different order than what is shown in a given flowchart. For example, again depending upon the technology involved, two operations shown in successive flowchart blocks may be performed in reverse order, as a single integrated step, concurrently, or in a manner at least partially overlapping in time.

A computer program product embodiment (“CPP embodiment” or “CPP”) is a term used in the present disclosure to describe any set of one, or more, storage media (also called “mediums”) collectively included in a set of one, or more, storage devices that collectively include machine readable code corresponding to instructions and/or data for performing computer operations specified in a given CPP claim. A “storage device” is any tangible device that can retain and store instructions for use by a computer processor. Without limitation, the computer readable storage medium may be an electronic storage medium, a magnetic storage medium, an optical storage medium, an electromagnetic storage medium, a semiconductor storage medium, a mechanical storage medium, or any suitable combination of the foregoing. Some known types of storage devices that include these mediums include: diskette, hard disk, random access memory (RAM), read-only memory (ROM), erasable programmable read-only memory (EPROM or Flash memory), static random access memory (SRAM), compact disc read-only memory (CD-ROM), digital versatile disk (DVD), memory stick, floppy disk, mechanically encoded device (such as punch cards or pits/lands formed in a major surface of a disc) or any suitable combination of the foregoing. A computer readable storage medium, as that term is used in the present disclosure, is not to be construed as storage in the form of transitory signals per se, such as radio waves or other freely propagating electromagnetic waves, electromagnetic waves propagating through a waveguide, light pulses passing through a fiber optic cable, electrical signals communicated through a wire, and/or other transmission media. As will be understood by those of skill in the art, data is typically moved at some occasional points in time during normal operations of a storage device, such as during access, de-fragmentation or garbage collection, but this does not render the storage device as transitory because the data is not transitory while it is stored.

Computing environment 100 contains an example of an environment for the execution of at least some of the computer code involved in performing a method for capturing distinct higher order interactions relevant to a biological inference 150. In addition to block 150, computing environment 100 includes, for example, computer 101, wide area network (WAN) 102, end user device (EUD) 103, remote server 104, public cloud 105, and private cloud 106. In this embodiment, computer 101 includes processor set 110 (including processing circuitry 120 and cache 121), communication fabric 111, volatile memory 112, persistent storage 113 (including operating system 122 and block 150, as identified above), peripheral device set 114 (including user interface (UI), device set 123, storage 124, and Internet of Things (IoT) sensor set 125), and network module 115. Remote server 104 includes remote database 130. Public cloud 105 includes gateway 140, cloud orchestration module 141, host physical machine set 142, virtual machine set 143, and container set 144.

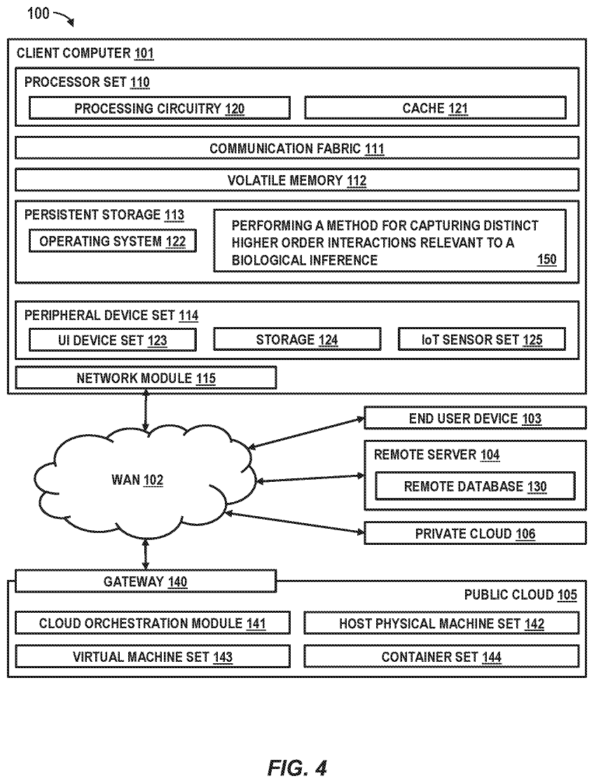

COMPUTER 101 may take the form of a desktop computer, laptop computer, tablet computer, smart phone, smart watch or other wearable computer, mainframe computer, quantum computer or any other form of computer or mobile device now known or to be developed in the future that is capable of running a program, accessing a network or querying a database, such as remote database 130. As is well understood in the art of computer technology, and depending upon the technology, performance of a computer-implemented method may be distributed among multiple computers and/or between multiple locations. On the other hand, in this presentation of computing environment 100, detailed discussion is focused on a single computer, specifically computer 101, to keep the presentation as simple as possible. Computer 101 may be located in a cloud, even though it is not shown in a cloud in FIG. 4. On the other hand, computer 101 is not required to be in a cloud except to any extent as may be affirmatively indicated.

PROCESSOR SET 110 includes one, or more, computer processors of any type now known or to be developed in the future. Processing circuitry 120 may be distributed over multiple packages, for example, multiple, coordinated integrated circuit chips. Processing circuitry 120 may implement multiple processor threads and/or multiple processor cores. Cache 121 is memory that is located in the processor chip package(s) and is typically used for data or code that should be available for rapid access by the threads or cores running on processor set 110. Cache memories are typically organized into multiple levels depending upon relative proximity to the processing circuitry. Alternatively, some, or all, of the cache for the processor set may be located “off chip.” In some computing environments, processor set 110 may be designed for working with qubits and performing quantum computing.

Computer readable program instructions are typically loaded onto computer 101 to cause a series of operational steps to be performed by processor set 110 of computer 101 and thereby effect a computer-implemented method, such that the instructions thus executed will instantiate the methods specified in flowcharts and/or narrative descriptions of computer-implemented methods included in this document (collectively referred to as “the inventive methods”). These computer readable program instructions are stored in various types of computer readable storage media, such as cache 121 and the other storage media discussed below. The program instructions, and associated data, are accessed by processor set 110 to control and direct performance of the inventive methods. In computing environment 100, at least some of the instructions for performing the inventive methods may be stored in block 150 in persistent storage 113.

COMMUNICATION FABRIC 111 is the signal conduction paths that allow the various components of computer 101 to communicate with each other. Typically, this fabric is made of switches and electrically conductive paths, such as the switches and electrically conductive paths that make up busses, bridges, physical input/output ports and the like. Other types of signal communication paths may be used, such as fiber optic communication paths and/or wireless communication paths.

VOLATILE MEMORY 112 is any type of volatile memory now known or to be developed in the future. Examples include dynamic type random access memory (RAM) or static type RAM. Typically, the volatile memory is characterized by random access, but this is not required unless affirmatively indicated. In computer 101, the volatile memory 112 is located in a single package and is internal to computer 101, but, alternatively or additionally, the volatile memory may be distributed over multiple packages and/or located externally with respect to computer 101.

PERSISTENT STORAGE 113 is any form of non-volatile storage for computers that is now known or to be developed in the future. The non-volatility of this storage means that the stored data is maintained regardless of whether power is being supplied to computer 101 and/or directly to persistent storage 113. Persistent storage 113 may be a read only memory (ROM), but typically at least a portion of the persistent storage allows writing of data, deletion of data and re-writing of data. Some familiar forms of persistent storage include magnetic disks and solid state storage devices. Operating system 122 may take several forms, such as various known proprietary operating systems or open source Portable Operating System Interface type operating systems that employ a kernel. The code included in block 150 typically includes at least some of the computer code involved in performing the inventive methods.

PERIPHERAL DEVICE SET 114 includes the set of peripheral devices of computer 101. Data communication connections between the peripheral devices and the other components of computer 101 may be implemented in various ways, such as Bluetooth connections, Near-Field Communication (NFC) connections, connections made by cables (such as universal serial bus (USB) type cables), insertion type connections (for example, secure digital (SD) card), connections made though local area communication networks and even connections made through wide area networks such as the internet. In various embodiments, UI device set 123 may include components such as a display screen, speaker, microphone, wearable devices (such as goggles and smart watches), keyboard, mouse, printer, touchpad, game controllers, and haptic devices. Storage 124 is external storage, such as an external hard drive, or insertable storage, such as an SD card. Storage 124 may be persistent and/or volatile. In some embodiments, storage 124 may take the form of a quantum computing storage device for storing data in the form of qubits. In embodiments where computer 101 is required to have a large amount of storage (for example, where computer 101 locally stores and manages a large database) then this storage may be provided by peripheral storage devices designed for storing very large amounts of data, such as a storage area network (SAN) that is shared by multiple, geographically distributed computers. IoT sensor set 125 is made up of sensors that can be used in Internet of Things applications. For example, one sensor may be a thermometer and another sensor may be a motion detector.

NETWORK MODULE 115 is the collection of computer software, hardware, and firmware that allows computer 101 to communicate with other computers through WAN 102. Network module 115 may include hardware, such as modems or Wi-Fi signal transceivers, software for packetizing and/or de-packetizing data for communication network transmission, and/or web browser software for communicating data over the internet. In some embodiments, network control functions and network forwarding functions of network module 115 are performed on the same physical hardware device. In other embodiments (for example, embodiments that utilize software-defined networking (SDN)), the control functions and the forwarding functions of network module 115 are performed on physically separate devices, such that the control functions manage several different network hardware devices. Computer readable program instructions for performing the inventive methods can typically be downloaded to computer 101 from an external computer or external storage device through a network adapter card or network interface included in network module 115.

WAN 102 is any wide area network (for example, the internet) capable of communicating computer data over non-local distances by any technology for communicating computer data, now known or to be developed in the future. In some embodiments, the WAN may be replaced and/or supplemented by local area networks (LANs) designed to communicate data between devices located in a local area, such as a Wi-Fi network. The WAN and/or LANs typically include computer hardware such as copper transmission cables, optical transmission fibers, wireless transmission, routers, firewalls, switches, gateway computers and edge servers.

END USER DEVICE (EUD) 103 is any computer system that is used and controlled by an end user (for example, a customer of an enterprise that operates computer 101), and may take any of the forms discussed above in connection with computer 101. EUD 103 typically receives helpful and useful data from the operations of computer 101. For example, in a hypothetical case where computer 101 is designed to provide a recommendation to an end user, this recommendation would typically be communicated from network module 115 of computer 101 through WAN 102 to EUD 103. In this way, EUD 103 can display, or otherwise present, the recommendation to an end user. In some embodiments, EUD 103 may be a client device, such as thin client, heavy client, mainframe computer, desktop computer and so on.

REMOTE SERVER 104 is any computer system that serves at least some data and/or functionality to computer 101. Remote server 104 may be controlled and used by the same entity that operates computer 101. Remote server 104 represents the machine(s) that collects and store helpful and useful data for use by other computers, such as computer 101. For example, in a hypothetical case where computer 101 is designed and programmed to provide a recommendation based on historical data, then this historical data may be provided to computer 101 from remote database 130 of remote server 104.

PUBLIC CLOUD 105 is any computer system available for use by multiple entities that provides on-demand availability of computer system resources and/or other computer capabilities, especially data storage (cloud storage) and computing power, without direct active management by the user. Cloud computing typically leverages sharing of resources to achieve coherence and economies of scale. The direct and active management of the computing resources of public cloud 105 is performed by the computer hardware and/or software of cloud orchestration module 141. The computing resources provided by public cloud 105 are typically implemented by virtual computing environments that run on various computers making up the computers of host physical machine set 142, which is the universe of physical computers in and/or available to public cloud 105. The virtual computing environments (VCEs) typically take the form of virtual machines from virtual machine set 143 and/or containers from container set 144. It is understood that these VCEs may be stored as images and may be transferred among and between the various physical machine hosts, either as images or after instantiation of the VCE. Cloud orchestration module 141 manages the transfer and storage of images, deploys new instantiations of VCEs and manages active instantiations of VCE deployments. Gateway 140 is the collection of computer software, hardware, and firmware that allows public cloud 105 to communicate through WAN 102.

Some further explanation of virtualized computing environments (VCEs) will now be provided. VCEs can be stored as “images.” A new active instance of the VCE can be instantiated from the image. Two familiar types of VCEs are virtual machines and containers. A container is a VCE that uses operating-system-level virtualization. This refers to an operating system feature in which the kernel allows the existence of multiple isolated user-space instances, called containers. These isolated user-space instances typically behave as real computers from the point of view of programs running in them. A computer program running on an ordinary operating system can utilize all resources of that computer, such as connected devices, files and folders, network shares, CPU power, and quantifiable hardware capabilities. However, programs running inside a container can only use the contents of the container and devices assigned to the container, a feature which is known as containerization.

PRIVATE CLOUD 106 is similar to public cloud 105, except that the computing resources are only available for use by a single enterprise. While private cloud 106 is depicted as being in communication with WAN 102, in other embodiments a private cloud may be disconnected from the internet entirely and only accessible through a local/private network. A hybrid cloud is a composition of multiple clouds of different types (for example, private, community or public cloud types), often respectively implemented by different vendors. Each of the multiple clouds remains a separate and discrete entity, but the larger hybrid cloud architecture is bound together by standardized or proprietary technology that enables orchestration, management, and/or data/application portability between the multiple constituent clouds. In this embodiment, public cloud 105 and private cloud 106 are both part of a larger hybrid cloud.

One or more embodiments described herein can utilize machine learning techniques to perform tasks. More specifically, one or more embodiments described herein can incorporate and utilize rule-based decision making and artificial intelligence (AI) reasoning to accomplish the various operations described herein, namely containers. A container is a VCE that uses operating-system-level virtualization. This refers to an operating system feature in which the kernel allows the existence of multiple isolated user-space instances, called containers. These isolated user-space instances typically behave as real computers from the point of view of programs running in them. A computer program running on an ordinary operating system can utilize all resources of that computer, such as connected devices, files and folders, network shares, CPU power, and quantifiable hardware capabilities. However, programs running inside a container can only use the contents of the container and devices assigned to the container, a feature which is known as containerization.

ANNs can be embodied as so-called “neuromorphic” systems of interconnected processor elements that act as simulated “neurons” and exchange “messages” between each other in the form of electronic signals. Similar to the so-called “plasticity” of synaptic neurotransmitter connections that carry messages between biological neurons, the connections in ANNs that carry electronic messages between simulated neurons are provided with numeric weights that correspond to the strength or weakness of a given connection. The weights can be adjusted and tuned based on experience, making ANNs adaptive to inputs and capable of learning. For example, an ANN for handwriting recognition is defined by a set of input neurons that can be activated by the pixels of an input image. After being weighted and transformed by a function determined by the network's designer, the activation of these input neurons are then passed to other downstream neurons, which are often referred to as “hidden” neurons. This process is repeated until an output neuron is activated. The activated output neuron determines which character was input. It should be appreciated that these same techniques can be applied in the case of containers. A container is a VCE that uses operating-system-level virtualization. This refers to an operating system feature in which the kernel allows the existence of multiple isolated user-space instances, called containers. These isolated user-space instances typically behave as real computers from the point of view of programs running in them. A computer program running on an ordinary operating system can utilize all resources of that computer, such as connected devices, files and folders, network shares, CPU power, and quantifiable hardware capabilities. However, programs running inside a container can only use the contents of the container and devices assigned to the container, a feature which is known as containerization.

In accordance with an embodiment, the invention provides a method for capturing distinct higher order interactions through the calculation of cumulants from Ising Hamiltonian parameters, where the distinct higher order interactions relevant to biological inferences is shown, and includes gathering a multi-nomic dataset 202 which may include data related to Genomes, Transcriptome, Proteome, Metabolome, Phenome, etc. The multi-nomic dataset is then processed to identify higher dimensional interactions (<fifth order cumulants) where the higher dimensional interactions may be further processed to determine information regarding a phenotype of interest. Two examples may include processing the higher dimensional interactions to determine information regarding a Cumulant Based Network Analysis (CuNA) and/or a Cumulant Based Drug Response (CuRes) Therapy. It should be appreciated that the invention may be used in conjunction with various tools and any machine learning methods may be applied as a downstream task after obtaining the cumulants. Moreover, the upstream data may be processed to compute cumulants having smaller set features from the data to accelerate its computation. Accordingly, standard machine learning libraries such as scikit-learn, etc., scientific programming libraries such as scipy, etc., or linear algebra libraries such as LAPACK, etc. can all be used in conjunction with the invention.

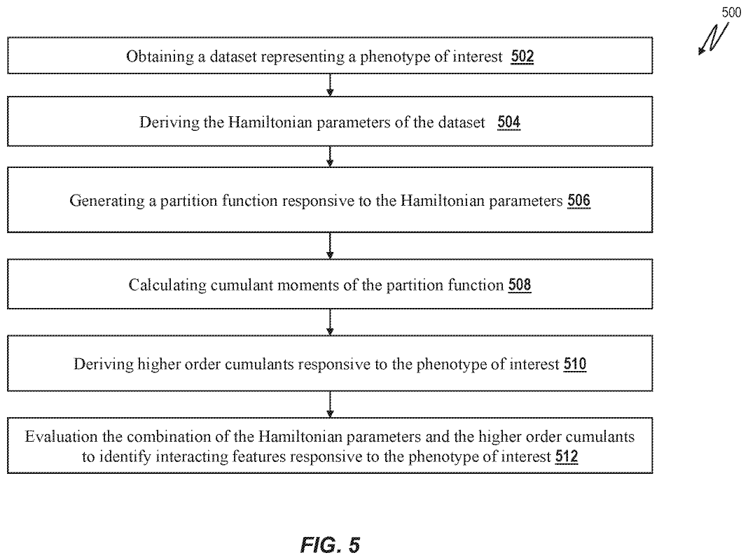

Referring to FIG. 5, an operational block diagram illustrating a method 500 for capturing distinct higher order interactions of a dataset relevant to a biological inference is shown and includes obtaining a dataset representing a phenotype of interest, such as which genetic alterations impact drug response therapy, as shown in operational block 502. The Hamiltonian parameters of the dataset are derived, as shown in operational block 504, where the Hamiltonian parameters embody a description of the interaction between biological state variables. It should be appreciated that the parameters include covariances of the Hamiltonian parameters and other internal components, thereby resulting in covariances among the cumulants. In one embodiment, the Hamiltonian parameters may be derived by using Machine Language (ML)/Artificial Intelligence (AI)/Regression approach. While in another embodiment the Hamiltonian parameters may be derived using a brute-force moments (i.e., third cumulant) computation approach where the results of the brute-force moments computation are optimized using an optimization procedure to obtain the Hamiltonian parameters, such as Newton's method (i.e., Newton-Raphson method), the Secant method, etc.

The partition function of the Hamiltonian parameters is generated and evaluated from its roots, as shown in operational block 506 and the cumulant moments of the partition function (i.e., zeros of the partition function) of the Hamiltonian parameters are computed, as shown in operational block 508. In an embodiment, the partition function may be analyzed to determine the complex roots (such as, for example, Yang-Lee complex roots) of the partition function, where the complex roots of the partition function among the internal components are used to facilitate cumulant computations. The higher order cumulants are then computed by taking the derivatives of the partition function, as shown in operational block 510, wherein the higher order cumulants (4^{th} order cumulants and above) are responsive to the partition function. The combination of the Hamiltonian parameters and the specific cumulants (i.e., redescriptions) are then evaluated to identify biological relationships which indicate interacting features responsive to and/or driving the phenotype of interest (e.g. therapeutic response, patient prognosis, disease risk, etc.), as shown in operational block 512. In an embodiment, threshold parameters may be created and used to identify the statistical significance of the Hamiltonian Parameters or various other desired characteristics of the Hamiltonian Parameters, and the cumulants may be tested and evaluated and may be used to identify higher order interactions relevant to biological inferences. The threshold parameters may also be used to limit combinations of the biological inferences of interest, to identify interactions relevant to the biological inferences of interest and/or to indicate a stopping condition parameter, such as when a statistically significant result is obtained. It should be appreciated that one or more threshold parameters may be predefined and may be dependent upon the situation. For example, in one embodiment, a statistically significant result may be a result that exceed a predefined p-value threshold parameter of 0.05 for some specific tests. In other embodiments, one or more threshold parameters may be based upon a Bonferroni corrected/adjusted p-value, when the p-value includes a plurality of hypotheses. In still yet other embodiments, one or more threshold parameters may be responsive to a parameter(s) that is tested over a range of thresholds/conditions (similar to various polygenic risk score analysis approaches). This increases the robustness of the invention.

It should be appreciated that the method of the invention allows the parameters of the generating function (i.e., Hamiltonian) to be ‘learned’ by using an optimization procedure (such as Newton-Raphson) to compute the Hamiltonian parameters from low order cumulants. Thus, biological insight is derived from both the parameter values of the Hamiltonian and specific cumulants. The method of the invention further uses linear regression from the dataset to obtain Hamiltonians including they higher-order interactions that are relevant to biological inferences. Joint cumulants may be extracted from the data using directional derivative derived scalar cumulants, where the directional derivative derived scalar cumulants may be determined using directional partition function roots.

Thus, the method of the invention uses Hamiltonian parameters and cumulants to identify significant redescriptions as interacting groups of state variables, where the Hamiltonian parameters describe the coupling of biological state variables. The partition function is represented in terms of its roots and allows for the computation of cumulants that describe biological state variable interactions. Thus, the method of the invention allows for biomarker discovery enabled through high-order cumulants that represent complex interactions among and between sets of features and for learnable parameters from the Hamiltonians to find cumulants, where the Hamiltonian represents biological systems.

It should be appreciated that because many diseases are the outcome of multiple biological signaling and phenotype(s), comorbidities, as a synergy of multiple diseases, is an example of why these interactions should be considered. Unfortunately, these interactions are often complex and difficult to discover. For example, inflammatory diseases typically have well-recognized clinical patterns that include multiple organs and many disease states in the intestine are mirrored in the brain, or shared between diseases, such as Crohn's disease (a major form of IBD) and Parkinson Disease. And, it is known that dysfunction of APOA4 expression can drive AD, IBD and heart disease. Therefore, in an embodiment, the invention may be used to compute cumulants in higher order to extract these cryptic interactions to discover common elements that exist between diseases which can be used to define new medical practice and/or more effective clinical procedures/actions.

Various embodiments of the invention are described herein with reference to the related drawings. Alternative embodiments of the invention can be devised without departing from the scope of this invention. Various connections and positional relationships (e.g., over, below, adjacent, etc.) are set forth between elements in the following description and in the drawings. These connections and/or positional relationships, unless specified otherwise, can be direct or indirect, and the present invention is not intended to be limiting in this respect. Accordingly, a coupling of entities can refer to either a direct or an indirect coupling, and a positional relationship between entities can be a direct or indirect positional relationship. Moreover, the various tasks and process steps described herein can be incorporated into a more comprehensive procedure or process having additional steps or functionality not described in detail herein.

For the sake of brevity, conventional techniques related to making and using aspects of the invention may or may not be described in detail herein. In particular, various aspects of computing systems and specific computer programs to implement the various technical features described herein are well known. Accordingly, in the interest of brevity, many conventional implementation details are only mentioned briefly herein or are omitted entirely without providing the well-known system and/or process details.

In some embodiments, various functions or acts can take place at a given location and/or in connection with the operation of one or more apparatuses or systems. In some embodiments, a portion of a given function or act can be performed at a first device or location, and the remainder of the function or act can be performed at one or more additional devices or locations.

The terminology used herein is for the purpose of describing particular embodiments only and is not intended to be limiting. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, element components, and/or groups thereof.

The corresponding structures, materials, acts, and equivalents of all means or step plus function elements in the claims below are intended to include any structure, material, or act for performing the function in combination with other claimed elements as specifically claimed. The present disclosure has been presented for purposes of illustration and description, but is not intended to be exhaustive or limited to the form disclosed. Many modifications and variations will be apparent to those of ordinary skill in the art without departing from the scope and spirit of the disclosure. The embodiments were chosen and described in order to best explain the principles of the disclosure and the practical application, and to enable others of ordinary skill in the art to understand the disclosure for various embodiments with various modifications as are suited to the particular use contemplated.

The diagrams depicted herein are illustrative. There can be many variations to the diagram or the steps (or operations) described therein without departing from the spirit of the disclosure. For instance, the actions can be performed in a differing order or actions can be added, deleted or modified. Also, the term “coupled” describes having a signal path between two elements and does not imply a direct connection between the elements with no intervening elements/connections therebetween. All of these variations are considered a part of the present disclosure.

The following definitions and abbreviations are to be used for the interpretation of the claims and the specification. As used herein, the terms “comprises,” “comprising,” “includes,” “including,” “has,” “having,” “contains” or “containing,” or any other variation thereof, are intended to cover a non-exclusive inclusion. For example, a composition, a mixture, process, method, article, or apparatus that comprises a list of elements is not necessarily limited to only those elements but can include other elements not expressly listed or inherent to such composition, mixture, process, method, article, or apparatus.

Additionally, the term “exemplary” is used herein to mean “serving as an example, instance or illustration.” Any embodiment or design described herein as “exemplary” is not necessarily to be construed as preferred or advantageous over other embodiments or designs. The terms “at least one” and “one or more” are understood to include any integer number greater than or equal to one, i.e. one, two, three, four, etc. The terms “a plurality” are understood to include any integer number greater than or equal to two, i.e. two, three, four, five, etc. The term “connection” can include both an indirect “connection” and a direct “connection.”

The terms “about,” “substantially,” “approximately,” and variations thereof, are intended to include the degree of error associated with measurement of the particular quantity based upon the equipment available at the time of filing the application. For example, “about” can include a range of +8% or 5%, or 2% of a given value.

The present invention may be a system, a method, and/or a computer program product at any possible technical detail level of integration. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instruction by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments described herein. Moreover, the embodiments or parts of the embodiments may be combined in whole or in part without departing from the scope of the invention.

What is claimed is:

1. A method for capturing distinct higher order interactions of a dataset relevant to biological inferences, the method comprising:
deriving Hamiltonian parameters for the dataset, wherein the dataset includes data responsive to a phenotype of interest and wherein the Hamiltonian parameters include Hamiltonian parameter covariances;
generating a partition function responsive to the Hamiltonian parameters;
calculating cumulant moments and complex roots of the partition function; and
deriving higher order cumulants using the Hamiltonian parameters, wherein the higher order cumulants are responsive to the complex roots and the phenotype of interest and include cumulant covariances.

2. The method of claim 1, wherein the phenotype of interest includes genetic alterations which impact drug response therapy.

3. The method of claim 1, wherein the Hamiltonian parameters are derived using a Machine Learning/Artificial Intelligence Regression model.

4. The method of claim 1, wherein the Hamiltonian parameters are derived by computing moments of the dataset and applying an optimization procedure to the moments.

5. The method of claim 4, wherein the optimization procedure is Newton-Raphson method.

6. The method of claim 1, wherein generating a partition function includes generating the partition function from the complex roots of the partition function, wherein the partition function is responsive to the Hamiltonian parameters.

7. The method of claim 1,
wherein calculating cumulant moments of the partition function includes calculating 3^{rd} order cumulants of the partition function, and
wherein deriving the higher order cumulants includes deriving 4^{th} order cumulants and higher.

8. The method of claim 1, further comprising evaluating a statistical significance of the Hamiltonian parameters and cumulants, wherein the statistical significance is used to identify higher order interactions relevant to biological inferences, to limit combinations of the biological inferences of interest and to indicate when a result exceeds a predefined threshold parameter.

9. A computing system, comprising:
a processor configured to perform operations for capturing distinct higher order interactions of a dataset relevant to a biological inference, the operations comprising:
deriving Hamiltonian parameters for the dataset, wherein the dataset includes data responsive to a phenotype of interest and wherein the Hamiltonian parameters include Hamiltonian parameter covariances;
generating a partition function responsive to the Hamiltonian parameters;
calculating cumulant moments and complex roots of the partition function; and
deriving higher order cumulants using the Hamiltonian parameters, wherein the higher order cumulants are responsive to the complex roots and the phenotype of interest and include cumulant covariances.

10. The computing system of claim 9, wherein the phenotype of interest includes genetic alterations which impact drug response therapy.

11. The computing system of claim 9, wherein the Hamiltonian parameters are derived using a Machine Learning/Artificial Intelligence Regression model.

12. The computing system of claim 9, wherein the Hamiltonian parameters are derived by computing moments of the dataset and applying an optimization procedure to the moments.

13. The computing system of claim 12, wherein the optimization procedure is Newton-Raphson method.

14. The computing system of claim 9, wherein generating a partition function includes generating the partition function from the complex roots of the partition function, wherein the partition function is responsive to the Hamiltonian parameters.

15. The computing system of claim 9,
wherein calculating cumulant moments of the partition function includes calculating 3^{rd} order cumulants of the partition function; and
wherein deriving the higher order cumulants includes deriving 4^{th} order cumulants and higher.

16. The computing system of claim 9, further comprising evaluating a statistical significance of the Hamiltonian parameters and cumulants, wherein the statistical significance is used to identify higher order interactions relevant to biological inferences, to limit combinations of the biological inferences of interest and to indicate when a result exceeds a predefined threshold parameter.

17. A computer program product comprising a computer readable storage medium having program instructions embodied therewith, the program instructions executable by a processor to cause the processor to perform operations for capturing distinct higher order interactions of a dataset relevant to a biological inference, the operations comprising:
deriving Hamiltonian parameters for the dataset, wherein the dataset includes data responsive to a phenotype of interest and wherein the Hamiltonian parameters include Hamiltonian parameter covariances;
generating a partition function responsive to the Hamiltonian parameters;
calculating cumulant moments and complex roots of the partition function; and
deriving higher order cumulants using the Hamiltonian parameters, wherein the higher order cumulants are responsive to the complex roots and the phenotype of interest and include cumulant covariances.

18. The computer program product of claim 17, wherein the phenotype of interest includes genetic alterations which impact drug response therapy.

19. The computer program product of claim 17, wherein the Hamiltonian parameters are derived using a Machine Learning/Artificial Intelligence Regression model.

20. The computer program product of claim 17, wherein the Hamiltonian parameters are derived by computing moments of the dataset and applying an optimization procedure to the moments.

21. The computer program product of claim 20, wherein the optimization procedure is Newton-Raphson method.

22. The computer program product of claim 17, wherein generating a partition function includes generating the partition function from the complex roots of the partition function, wherein the partition function is responsive to the Hamiltonian parameters.

23. The computer program product of claim 17, wherein
wherein calculating cumulant moments of the partition function includes calculating 3^{rd} order cumulants of the partition function; and
wherein deriving the higher order cumulants includes deriving 4^{th} order cumulants and higher.

24. A method for capturing distinct higher order interactions of a dataset relevant to a biological inference, the method comprising:
obtaining the dataset, wherein the dataset is responsive to a phenotype of interest;
deriving Hamiltonian parameters for the dataset using a Machine Learning/Artificial Intelligence Regression model and wherein the Hamiltonian parameters include Hamiltonian parameter covariances;
generating a partition function responsive to the Hamiltonian parameters;
calculating cumulant moments and complex roots of the partition function; and
deriving higher order cumulants using the Hamiltonian parameters, wherein the higher order cumulants are responsive to the complex roots and the phenotype of interest and include cumulant covariances.

25. A method for capturing distinct higher order interactions of a dataset relevant to a biological inference, the method comprising:
obtaining the dataset, wherein the dataset is responsive to a phenotype of interest;
deriving Hamiltonian parameters for the dataset by computing moments of the dataset and applying an optimization procedure to the moments and wherein the Hamiltonian parameters include Hamiltonian parameter covariances;
generating a partition function responsive to the Hamiltonian parameters;
calculating cumulant moments and complex roots of the partition function; and
deriving higher order cumulants using the Hamiltonian parameters, wherein the higher order cumulants are responsive to the complex roots and the phenotype of interest and include cumulant covariances.
